(19) United States
(12) Patent Application Publication
(10) Pub. No.: US 2024/0242822 A1
(43) Pub. Date: Jul. 18, 2024

(54) METHODS AND SYSTEMS FOR PROACTIVE EQUIPMENT PLANNING AND PATIENT TRANSFER

(71) Applicant: KONINKLIJKE PHILIPS N.V., EINDHOVEN (NL)

(72) Inventors: PAUL FRANZ REDDER, NEWBERRY, FL (US); MICHAEL ANGELO GEMMATI JR, OVIEDO, FL (US); MARK SHIH-CHIEH LIN, WINTER PARK, FL (US)

(21) Appl. No.: 18/563,414

(22) PCT Filed: May 18, 2022
(86) PCT No.: PCT/EP2022/063363

§ 371 Date: Nov. 22, 2023

(51) Int. Cl. G16H 40/20; G06Q 10/0631; G16H 40/67

(52) U.S. Cl. CPC G16H 40/20; G06Q 10/0631; G16H 40/67

(57) ABSTRACT

A method (100) for transferring a patient from a first healthcare environment to a second healthcare environment, comprising: (i) providing (110) a composite network configured to obtain and communicate first information about one or more healthcare assets associated with the patient, and monitoring information about the patient: (ii) receiving (120) a request for the first information and the monitoring information: (iii) communicating (130) the first information and the monitoring information: (iv) configuring (140) one or more healthcare assets in the second healthcare-environment in preparation for a transfer: (v) determining (150) whether the second healthcare environment is ready for the transfer of the patient from the first healthcare environment: (vi) communicating (160) a signal indicating a result of the determining step: and (vii) receiving (180) the patient at the second healthcare environment, or delaying the patient from being transferred to the second healthcare environment.

### FIELD OF THE DISCLOSURE

The present disclosure is directed generally to methods and systems for efficiently and safely transferring a patient from a first healthcare environment to a second healthcare environment.

### BACKGROUND

During the course of healthcare treatment, a patient may experience or require many different healthcare settings, which requires the transfer of the patient from one healthcare setting to another setting. Often, a transfer involves many different requirements to ensure the safety and health of the patient. For example, a patient may be transferred from an emergency transport such as an ambulance to a second healthcare setting such as an emergency room. If the patient is being treated in the emergency transport, an efficient and safe transfer to the second healthcare setting can be accomplished by continuing that same treatment in the new setting. Thus, information about the treatment of the patient, the equipment being used and the equipment necessary for the treatment of the patient, as well as vital signs, demographic information, or other information about the patient can be communicated from the emergency transport to the second healthcare setting. Automated transfer of this information may facilitate communication and improve the care of the patient.

As another non-limiting example, a patient in a first healthcare setting or environment such as an emergency room, intensive care unit, or other care setting may require imaging. This may require transfer of the patient from the first healthcare setting to a second healthcare setting or environment in which the imaging equipment is located. However, such a transfer can create or involve many different complications. For example, a patient that is being treated with numerous pieces of equipment such as IVs, monitors, oxygen, and/or many other types of equipment may require some or all of that equipment in the imaging environment. Additionally, in specialized imaging environments such as an MRI environment, it is vital to the safety of the patient that non-MRI compatible equipment associated with the patient in the first environment is transferred to MRI-compatible equipment or no equipment in the second environment.

Scheduling of equipment and personnel is another example of a complication that can arise from the transfer of patients from one healthcare setting to another. Additionally, prescription medications given to the patient can be another complication that can arise from the transfer of patients from one healthcare setting to another, especially when infusion pumps are required. If a patient is transferred to a second healthcare setting when that setting is not prepared for the patient, this can result in inefficient utilization of equipment, space, and/or personnel. These issues can raise healthcare costs, among other issues. However, ensuring the efficient and safe transfer of a patient from one setting to another facilitates more efficient utilization of equipment, space, and personnel.

### SUMMARY OF THE DISCLOSURE

Accordingly, there is a continued need in the art for methods and systems that facilitate the safe and efficient transfer of a patient from a first healthcare environment to a second healthcare environment.

The present disclosure is directed to inventive methods and systems for patient transfer. Various embodiments and implementations herein are directed to a method and system configured for the safe and efficient transfer of a patient from a first healthcare environment to a second healthcare environment. A patient transfer system comprises a composite network configured to obtain and communicate: (i) first information about one or more healthcare assets associated with the patient in the first environment; and (ii) monitoring information about the patient. The system receives, at the first environment via the composite network, a request from the second healthcare environment for the first information and the monitoring information, while the patient is in the first healthcare environment. The first information and the monitoring information is communicated, via the composite network, from the first healthcare environment to the second healthcare environment. One or more healthcare assets in the second healthcare environment are configured, based on at least the received first information, in the second healthcare environment in preparation for a transfer of the patient. The patient transfer system determines whether the second healthcare environment is ready for the transfer of the patient from the first healthcare environment, wherein determining comprises a determination that the second healthcare environment comprises at least: (i) all necessary healthcare assets for the patient; and (ii) all necessary healthcare asset configurations for the patient. A signal is then communicated from the second healthcare environment to the first healthcare environment indicating a result of the determining step. When the second healthcare environment is determined to be ready for the transfer, the patient is received at the second healthcare environment. When the second healthcare environment is determined not to be ready for the transfer, the patient is delayed from being transferred to the second healthcare environment.

According to one aspect, a method for transferring a patient from a first healthcare environment to a second healthcare environment is provided. The method includes: (i) providing a composite network configured to obtain and communicate: first information about one or more healthcare assets associated with the patient in the first environment; and monitoring information about the patient; (ii) receiving, at the first environment via the composite network, a request from the second healthcare environment for the first information and the monitoring information, while the patient is in the first healthcare environment; (iii) communicating, via the composite network, the first information and the monitoring information from the first healthcare environment to the second healthcare environment; (iv) configuring, based on at least the received first information, one or more healthcare assets in the second healthcare environment in preparation for a transfer of the patient; (v) determining whether the second healthcare environment is ready for the transfer of the patient from the first healthcare environment, wherein determining comprises a determination that the second healthcare environment comprises at least: all necessary healthcare assets for the patient; and all necessary healthcare asset configurations for the patient; (vii) communicating, via the composite network, a signal from the second healthcare environment to the first healthcare environment indicating a result of the determining step; and (viii) receiving, when the second healthcare environment is determined to be ready for the transfer, the patient at the second healthcare environment, or delaying, when the second healthcare environment is determined not to be ready for the transfer, the patient from being transferred to the second healthcare environment.

According to an embodiment, the composite network is a single healthcare network configured to communicate the first information and the monitoring information from the first healthcare environment to the second healthcare environment.

According to an embodiment, the one or more healthcare assets associated with the patient in the first environment comprise medical equipment.

According to an embodiment, the step of configuring one or more healthcare assets in the second healthcare environment in preparation for a transfer of the patient comprises adjusting a setting of one or more pieces of medical equipment in the second healthcare environment.

According to an embodiment, the monitoring information about the patient comprises one or more vital signs and/or one or more healthcare treatments for the patient.

According to an embodiment, the step of receiving a request from the second healthcare environment for the first information and the monitoring information is in response to a predetermined schedule for the second environment.

According to an embodiment, the steps of receiving a request from the second healthcare environment for the first information and the monitoring information through the step of communicating a signal from the second healthcare environment to the first healthcare environment indicating a result of the determining step are automated without human intervention.

According to an embodiment, the method further includes the step of indicating to a healthcare provider in the first environment either: (i) that the second healthcare environment is ready for a patient transfer if the second healthcare environment is determined to be ready for the transfer; or (ii) that the second healthcare environment is not ready for a patient transfer if the second healthcare environment is determined to not be ready for the transfer.

According to another aspect is a system configured to enable transfer of a patient from a first healthcare environment to a second healthcare environment. The system includes: a first healthcare environment comprising first information comprising information about one or more healthcare assets associated with the patient in the first environment and monitoring information comprising health information about the patient; a second healthcare environment; a composite network configured to communicate information between the first healthcare environment and the second healthcare environment; and a processor configured to: (i) direct communication, via the composite network, of the first information and the monitoring information from the first healthcare environment to the second healthcare environment; (ii) direct configuration, based on the communicated first information, of one or more healthcare assets in the second healthcare environment in preparation for a transfer of the patient; (iii) determine whether the second healthcare environment is ready for the transfer of the patient from the first healthcare environment, wherein determining comprises a determination that the second healthcare environment comprises at least: all necessary healthcare assets for the patient; and all necessary healthcare asset configurations for the patient; and (iv) direct communication, via the composite network, of a signal from the second healthcare environment to the first healthcare environment indicating a result of the determination of readiness or non-readiness.

According to an embodiment, the system further includes a user interface configured to display, to a healthcare provider in the first environment either: (i) that the second healthcare environment is ready for a patient transfer if the second healthcare environment is determined to be ready for the transfer; or (ii) that the second healthcare environment is not ready for a patient transfer if the second healthcare environment is determined to not be ready for the transfer.

According to an embodiment, the composite network is a single healthcare network configured to communicate the first information and the monitoring information from the first healthcare environment to the second healthcare environment.

According to an embodiment, the processor is configured to direct communication, via the composite network, of the first information and the monitoring information in response to a predetermined schedule for the first environment and/or the second environment.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for transferring a patient from a first healthcare environment to a second healthcare environment, in accordance with an embodiment.

FIG. 2 is a schematic representation of a patient transfer system, in accordance with an embodiment.

FIG. 3 is a schematic representation of a patient transfer system, in accordance with an embodiment.

FIG. 4 is a schematic representation of a patient transfer system, in accordance with an embodiment.

### DETAILED DESCRIPTION OF EMBODIMENTS

The present disclosure describes various embodiments of a system and method for transferring a patient from a first healthcare environment to a second healthcare environment. More generally, Applicant has recognized and appreciated that it would be beneficial to provide a method and system to promote the safety of patients in the healthcare setting while facilitating the efficient use of healthcare resources. Accordingly, a patient transfer system is provided. The system comprises a composite network configured to obtain and communicate: (i) first information about one or more healthcare assets associated with the patient in the first environment; and (ii) monitoring information about the patient. The system receives, at the first environment via the composite network, a request from the second healthcare environment for the first information and the monitoring information, while the patient is in the first healthcare environment. The first information and the monitoring information is communicated, via the composite network, from the first healthcare environment to the second healthcare environment. One or more healthcare assets in the second healthcare environment are configured, based on at least the received first information, in the second healthcare environment in preparation for a transfer of the patient. The patient transfer system determines whether the second healthcare environment is ready for the transfer of the patient from the first healthcare environment, wherein determining comprises a determination that the second healthcare environment comprises at least: (i) all necessary healthcare assets for the patient; and (ii) all necessary healthcare asset configurations for the patient. A signal is then communicated from the second healthcare environment to the first healthcare environment indicating a result of the determining step. When the second healthcare environment is determined to be ready for the transfer, the patient is received at the second healthcare environment. When the second healthcare environment is determined not to be ready for the transfer, the patient is delayed from being transferred to the second healthcare environment.

Accordingly, the methods and systems described or otherwise envisioned herein supports the safe and efficient transfer of a patient from a first healthcare environment to a second healthcare environment. The methods and systems address and prevent the inefficiencies, potential hazards, and delays in providing care that arise from patient transfers using prior art methods and systems. Thus, the methods and systems described or otherwise envisioned herein promote the safety and care of patients while increasing the optimal utilization of healthcare resources and personnel.

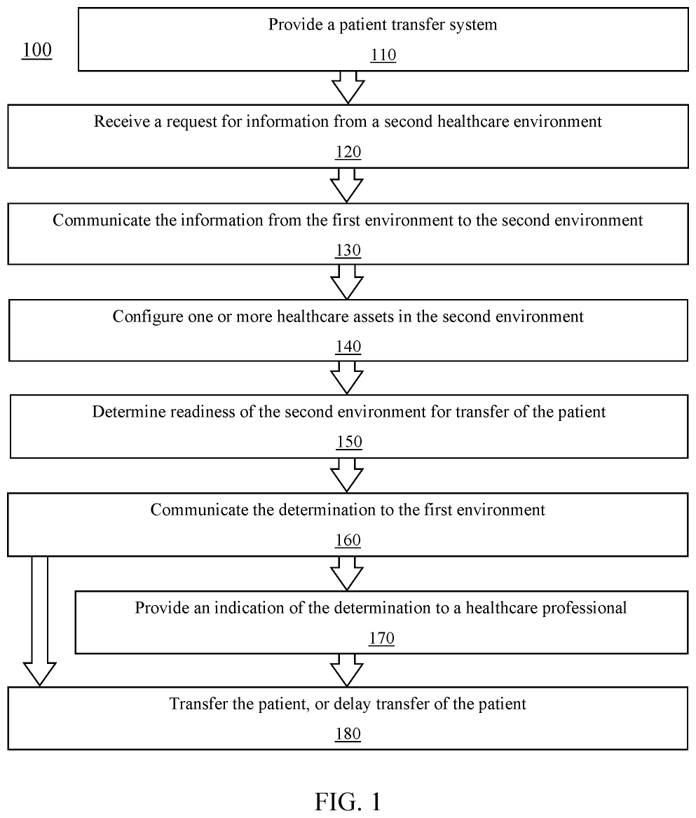

Referring to FIG. 1, in one embodiment, is a flowchart of a method 100 for transferring a patient from a first healthcare environment to a second healthcare environment using a patient transfer system. The methods described in connection with the figures are provided as examples only and shall be understood not to limit the scope of the disclosure. The patient transfer system can be any of the systems described or otherwise envisioned herein. The healthcare settings within which the patient is found or transferred can be any healthcare setting.

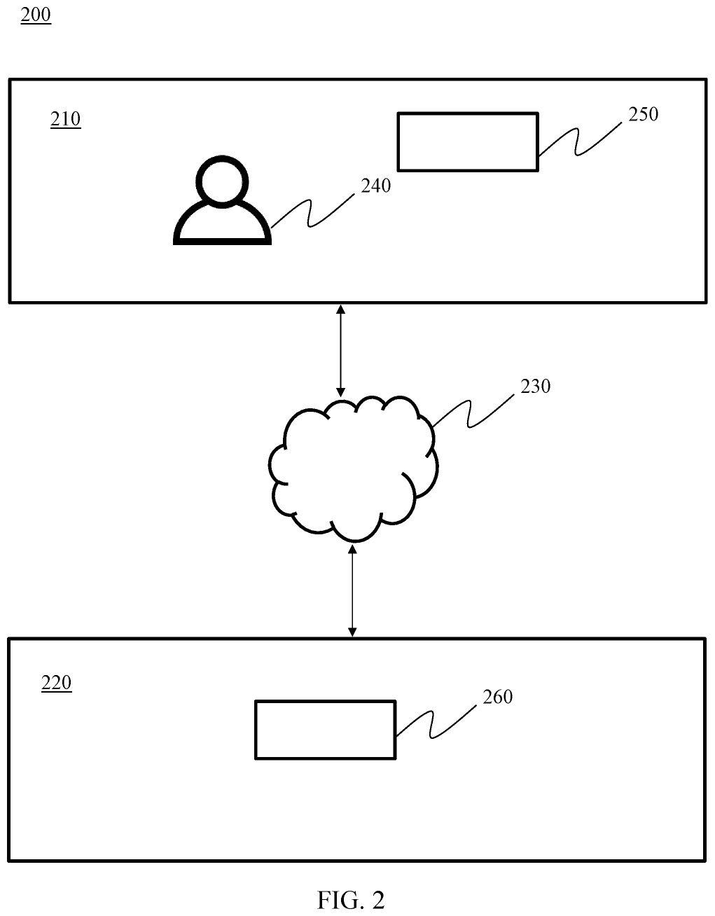

At step 110 of the method, according to an embodiment, a patient transfer system 200 is provided. Referring to an embodiment of a patient transfer system 200 as depicted in FIG. 2, for example, the system comprises a composite network 230 connecting a plurality of healthcare environments such as 210 and 220. For example, the patient transfer system may comprise a single healthcare setting such as a hospital, or may comprise multiple different healthcare settings or environments. According to an embodiment, the composite network 230 is a single network configured to communicate both information about healthcare assets, and monitoring or other health or medical record information about the patient. It will be understood that FIG. 2 constitutes, in some respects, an abstraction and that the actual organization of the components of the system 200 may be different and more complex than illustrated.

A first healthcare environment 210 of the patient transfer system may be any healthcare setting. For example, the first healthcare setting 210 may be an emergency transport such as an ambulance or may be a patient care setting such as a hospital room, emergency room, intensive care unit, or any other patient care setting.

According to an embodiment, the first healthcare setting comprises one or more healthcare assets 250. A healthcare asset can be any non-human device, equipment, monitor, or other thing or asset that is utilized in the care or treatment of a patient. For example, the healthcare asset can be an IV, vital sign monitor, oxygen providing element, ventilator, and/or any other piece of equipment or device. As another example, the healthcare asset could be care equipment such as imaging equipment like MRI, CT, x-ray equipment, and many other types of equipment. According to an embodiment, one or more of the healthcare assets 250 may comprise a setting or parameter that has been set or adjusted for the patient. For example, an IV may be configured to provide a specific medication or treatment at a specific rate or timing. A ventilator may be configured to provide ventilation according to a set of parameters. These configurations, settings, or parameters comprise a component of the first healthcare setting. The first healthcare setting may comprise many other elements, components, and/or personnel.

The first healthcare setting also comprises a patient 240 that is receiving care in the environment. According to an embodiment, the patient is being monitored by one or more healthcare assets configured to obtain information about the patient, such as vital sign monitoring or other information. As another example, the monitoring information about the patient may comprise treatment options such as a prescription or non-premedication, physical treatment, or other type of treatment. This monitoring information about the patient comprises a component of the first healthcare setting.

At step 120 of the method, according to an embodiment, the system receives, at a first healthcare environment 210 via the composite network 230, a request from a second healthcare environment 220 for: (i) first information about the one or more healthcare assets 250 associated with the patient 240 in the first environment; and (ii) monitoring information about the patient 240. This request is received from the second environment while the patient is in the first environment. According to an embodiment, the request is an automated request without human intervention or activation. For example, the request may be initiated by a predetermined schedule for the first and/or second healthcare environment.

As just one non-limiting example, the second healthcare environment 220 can be an environment which is highly utilized and thus comprises a schedule that accommodates use by numerous patients, such as an imaging environment. The second healthcare setting may comprise one or more healthcare assets 260. The imaging environment may optimize usage of the imaging equipment by utilizing a schedule. This schedule can interact or interface with the patient transfer system in order to request information about a schedule patient in advance of the patient's scheduled usage of the imaging environment. This may ensure that the patient, the first environment, and the second environment are prepared for the healthcare needs/requirements and imaging needs of the patient. This avoids inefficient use of the imaging environment. For example, a patient may be rescheduled to a new timeslot if one or more of the patient, the first environment, and the second environment are not prepared or otherwise suitable for the patient transfer.

According to another embodiment, the first healthcare environment sends the first information and/or monitoring information to the second healthcare environment without a request from the second environment. For example, the first environment may be an emergency transport such as an ambulance, and the second environment may be a hospital or other setting that will receive the patient. Thus, the emergency transport may send the first information and/or monitoring information to the receiving environment in anticipation of arrival of the patient at the hospital.

At step 130 of the method, according to an embodiment, the patient transfer system communicates the first information and the monitoring information from the first healthcare environment to the second healthcare environment via the composite network, in response to a request for information. Alternatively, the patient transfer system communicates the first information and the monitoring information in response to a command or other direction from the second environment to send the information to the first environment. The information can be stored in memory or any other data structure for immediate and/or later use by the system.

At step 140 of the method, according to an embodiment, one or more healthcare assets in the second healthcare environment are configured in preparation for transfer of the patient, based on at least the received first information. The configuration may also be based on the received monitoring information, among other information. Configuring a healthcare asset can comprise a wide variety of options, including but not limited to providing an asset, adjusting an asset, or otherwise making any provision or change of an asset. As a non-limiting example, configuring a healthcare asset based on the received first information in preparation for transfer of a patient may comprise, for example, ensuring all equipment necessary for the patient's treatment such as IVs and vital sign monitors are present within the second environment, and that the equipment is configured to meet the needs of the patient. For example, the patient may require specific medication or monitoring, and thus the second environment must be configured in advance to provide that specific required medication and monitoring.

At step 150 of the method, according to an embodiment, the patient transfer system determines whether the second healthcare environment is ready for the transfer of the patient from the first healthcare environment. This comprises a determination that the second healthcare environment comprises at least: (i) all necessary healthcare assets for the patient; and (ii) all necessary healthcare asset configurations for the patient. According to an embodiment, this determination may comprise a minimum threshold. For example, the information provided to the second environment about the patient may comprise both required assets and configurations, and desired but not necessary assets and configurations. Thus, the readiness of the second environment may comprise a determination that at least the required assets and configurations are available, or a determination that both the required and desired assets and configurations are available.

Determining readiness of the second healthcare environment for the arrival of the patient may comprise, for example, an evaluation of healthcare asset inventory within the second environment. This can be done manually or via an automated process. According to another embodiment, the readiness of the second healthcare environment is performed in whole or in part by a healthcare professional or other individual in the second environment. For example, the individual can survey, provide, adjust, or otherwise ensure all required and/or desired assets and configurations are available and/or set within the second environment.

Determining readiness of the second healthcare environment for the arrival of the patient may comprise, for example, a possible, estimated, or expected arrival time for the patient.

At step 160 of the method, according to an embodiment, the patient transfer system communicates, via the composite network, a signal from the second healthcare environment to the first healthcare environment indicating a result of the determining step. For example, the patient transfer system may send a signal to the first environment that the second environment is ready or will be ready to receive the patient. The signal can be any indication of readiness. For example, the indication of readiness may comprise a possible, estimated, or expected arrival time for the patient. As another example, the transfer system may send a signal to the first environment that the second environment is not ready or will not be ready to receive the patient. The signal can be any indication that the second environment is not ready. The signal of readiness or unpreparedness for receipt of the patient can be an automated signal, such as a response from the second environment that the necessary equipment is within—or not within—the inventory of the second environment. Alternatively, the signal of readiness or unpreparedness for receipt of the patient can be triggered or sent manually, such as in response to a manual determination by a healthcare professional that an asset is not available or not ready for receipt of the patient. According to an embodiment, a signal is communicated only if the signal comprises an indication of readiness, in which case the system will proceed with the transfer. Pursuant to this embodiment, the system may return to a prior step of the method if the signal comprises an indication of unpreparedness for a transfer, among other options.

At the first environment, a received signal of readiness may trigger one or more actions. For example, a signal that the second environment is ready for transfer of the patient may trigger the transfer of the patient, or trigger a signal to a healthcare professional in the first environment that the patient is ready for transfer. Many other actions are possible. At the first environment, a received signal that the second environment is not or will not be ready for transfer of the patient as expected or requested may trigger one or more actions. For example, a signal that the second environment is not ready for transfer of the patient may trigger a delay of the transfer, may trigger the transfer or analysis of a second patient, may adjust a schedule, or may trigger a signal to a healthcare professional in the first environment that the transfer is not ready. Many other actions are possible.

At step 170 of the method, according to an embodiment, the patient transfer system may provide an indication to a healthcare professional or other user that either: (i) the second healthcare environment is ready for a patient transfer if the second healthcare environment is determined to be ready for the transfer; or (ii) the second healthcare environment is not ready for a patient transfer if the second healthcare environment is determined to not be ready for the transfer. The indication can be any signal or indication of readiness or unreadiness. For example, the signal can be an indication on a patient monitor or other user interface, such as a message, icon, alert, sound, or any other signal or indication. The signal can be sent to any local or remote location required to notify a healthcare professional or other user. The user can be in the first healthcare environment, the second healthcare environment, or in any other healthcare environment.

At step 180 of the method, according to an embodiment, if the second healthcare environment is determined to be ready for the transfer, the second environment may receive the patient via the patient transfer. Alternatively, according to an embodiment, if the second healthcare environment is determined to not be ready for the transfer, the patient transfer system may delay or cancel the patient from being transferred to the second healthcare environment.

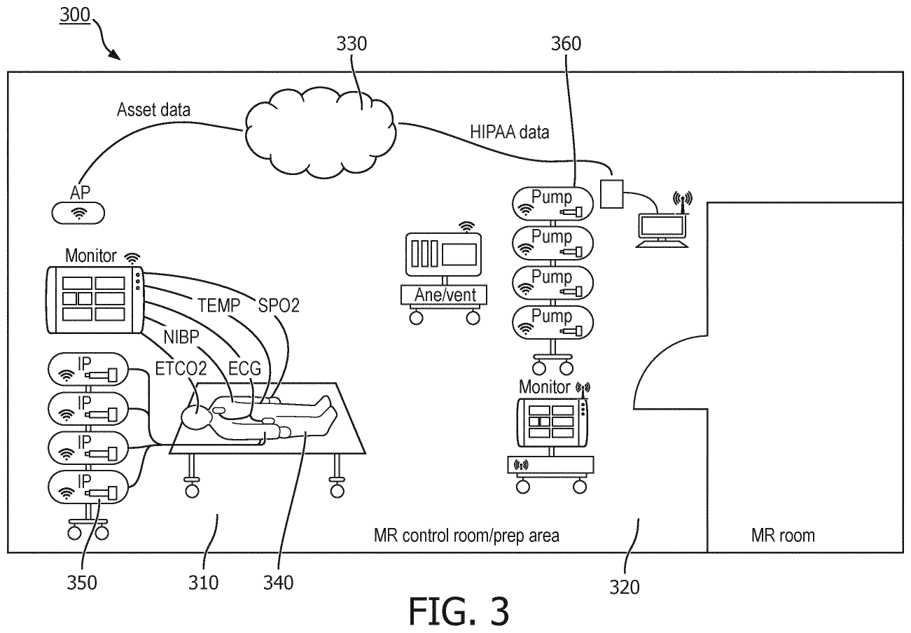

Referring to FIG. 3, in accordance with an embodiment, is a patient transfer system 300 comprising a composite network 330 communicating information between first healthcare environment 310 within which patient 340 is located, and second healthcare environment 320. In this non-limiting example, the patient is being transferred to an MRI imaging environment. According to an embodiment, composite network 330 is a single network configured to communicate both information about healthcare assets, and monitoring or other health or medical record information about the patient. It will be understood that FIG. 3 constitutes, in some respects, an abstraction and that the actual organization of the components of the system 300 may be different and more complex than illustrated.

The first healthcare environment 310 of the patient transfer system may be any healthcare setting. For example, the first healthcare setting 310 may be a patient care setting such as a hospital room, emergency room, intensive care unit, or any other patient care setting. The first healthcare setting comprises one or more healthcare assets 350, such as one or more IVs and one or more patient monitors. According to an embodiment, one or more of the healthcare assets 350 may comprise a setting or parameter that has been set or adjusted for the patient. For example, an IV may be configured to provide a specific medication or treatment at a specific rate or timing. These configurations, settings, or parameters comprise a component of the first healthcare setting. The first healthcare setting may comprise many other elements, components, and/or personnel not shown in FIG. 3.

The first healthcare setting also comprises a patient 340 that is receiving care in the environment. According to an embodiment, the patient is being monitored by one or more healthcare assets configured to obtain information about the patient, such as vital sign monitoring or other information. This monitoring information about the patient comprises a component of the first healthcare setting.

According to another embodiment, the first healthcare environment sends first information and/or monitoring information to the second healthcare environment in preparation for a transfer of the patient 340. According to an embodiment, the second healthcare environment 320 is an MRI environment, although the second environment can be any environment to which a patient may be transferred. The second healthcare environment comprises one or more healthcare assets 360 that may be configured, adjusted, or otherwise made suitable for the transfer and receipt of the patient 340. For example, as shown in FIG. 3, the second environment has received the first information and/or monitoring information transmitted via the composite network 330, and thus the plurality of healthcare assets 360 in the second environment, including a patient monitor and IVs, are available and configured in advance of the transfer of the patient. Preparation in advance of the transfer of the patient, in response to information transmitted via the composite network, optimizes usage of the imaging equipment by preventing the transfer of patients that are not ready for transfer, encouraging transfer of patients that are ready for transfer, and preventing transfer of patients when the second environment is not ready for the transfer, and via other possible mechanisms.

As an example of the patient transfer system, the first healthcare environment sends first information and/or monitoring information to the second healthcare environment in preparation for a transfer of the patient. According to an embodiment, the first healthcare environment is an emergency environment such as an accident scene, ambulance, or other emergency environment. According to an embodiment, the first healthcare setting may be looking for any second environment capable of caring for the patient currently in the emergency environment. Thus, for example, the first healthcare setting may be looking for an emergency room or other setting that comprises the medication, treatment, healthcare assets, or other equipment or items necessary for the care of the patient. The first healthcare setting may therefore communicate the first information and/or monitoring information to a plurality of second healthcare environments, receive information about capabilities from one or more of the plurality of second healthcare environments, and select a second healthcare environment based on the receive information. Preparation of the second healthcare environment in advance of the transfer of the patient, in response to information transmitted via the composite network, optimizes the patient's care in the emergency setting. The system prevents poor care of patients by preventing the transfer of patients when the second environment is not ready for the transfer.

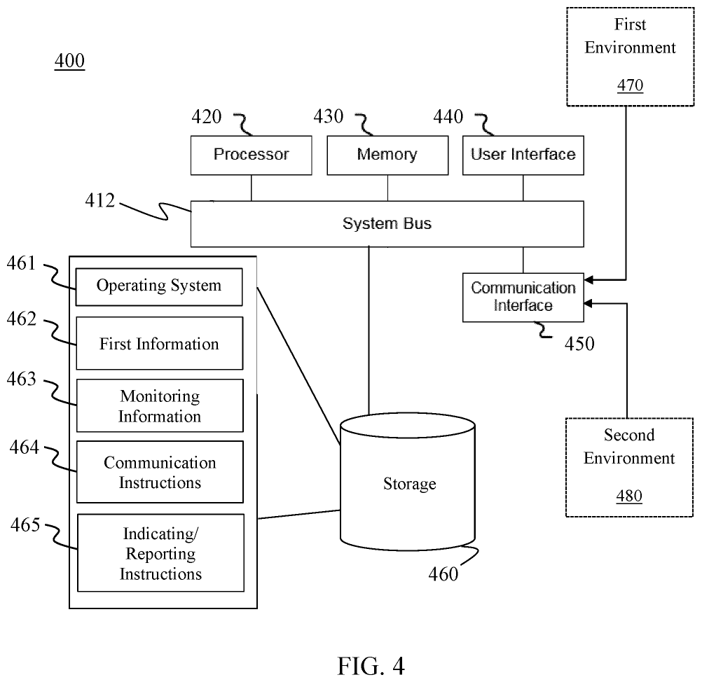

Referring to FIG. 4, in one embodiment, is a schematic representation of a patient transfer system 700. System 400 may be any of the systems described or otherwise envisioned herein, and may comprise any of the components described or otherwise envisioned herein. According to an embodiment, system 400 comprises one or more of a processor 420, memory 430, user interface 440, communications interface 450, and storage 460, interconnected via one or more system buses 412. It will be understood that FIG. 4 constitutes, in some respects, an abstraction and that the actual organization of the components of the system 400 may be different and more complex than illustrated.

According to an embodiment, system 400 comprises a processor 420 capable of executing instructions stored in memory 430 or storage 460 or otherwise processing data to, for example, perform one or more steps of the method. Processor 420 may be formed of one or multiple modules. Processor 420 may take any suitable form, including but not limited to a microprocessor, microcontroller, multiple microcontrollers, circuitry, field programmable gate array (FPGA), application-specific integrated circuit (ASIC), a single processor, or plural processors.

Memory 430 can take any suitable form, including a non-volatile memory and/or RAM. The memory 430 may include various memories such as, for example L1, L2, or L3 cache or system memory. As such, the memory 430 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 400. It will be apparent that, in embodiments where the processor implements one or more of the functions described herein in hardware, the software described as corresponding to such functionality in other embodiments may be omitted.

User interface 440 may include one or more devices for enabling communication with a user. The user interface can be any device or system that allows information to be conveyed and/or received, and may include a display, a mouse, and/or a keyboard for receiving user commands. In some embodiments, user interface 440 may include a command line interface or graphical user interface that may be presented to a remote terminal via communication interface 450. The user interface may be located with one or more other components of the system, or may located remote from the system and in communication via a wired and/or wireless communications network.

Communication interface 450 may include one or more devices for enabling communication with other hardware devices. For example, communication interface 450 may include a network interface card (NIC) configured to communicate according to the Ethernet protocol. Additionally, communication interface 450 may implement a TCP/IP stack for communication according to the TCP/IP protocols. Various alternative or additional hardware or configurations for communication interface 450 will be apparent.

According to an embodiment, communication interface may comprise or interact with a composite network connecting a plurality of healthcare environments such as first healthcare environment 470 and second healthcare environment 480. For example, the patient transfer system may comprise a single healthcare setting such as a hospital, or may comprise multiple different healthcare settings or environments. According to an embodiment, the composite network is a single network configured to communicate both information about healthcare assets, and monitoring or other health or medical record information about the patient.

Storage 460 may include one or more machine-readable storage media such as read-only memory (ROM), random-access memory (RAM), magnetic disk storage media, optical storage media, flash-memory devices, or similar storage media. In various embodiments, storage 460 may store instructions for execution by processor 420 or data upon which processor 420 may operate. For example, storage 460 may store an operating system 461 for controlling various operations of system 400.

It will be apparent that various information described as stored in storage 460 may be additionally or alternatively stored in memory 430. In this respect, memory 430 may also be considered to constitute a storage device and storage 460 may be considered a memory. Various other arrangements will be apparent. Further, memory 430 and storage 460 may both be considered to be non-transitory machine-readable media. As used herein, the term non-transitory will be understood to exclude transitory signals but to include all forms of storage, including both volatile and non-volatile memories.

While system 400 is shown as including one of each described component, the various components may be duplicated in various embodiments. For example, processor 420 may include multiple microprocessors that are configured to independently execute the methods described herein or are configured to perform steps or subroutines of the methods described herein such that the multiple processors cooperate to achieve the functionality described herein. Further, where one or more components of system 400 is implemented in a cloud computing system, the various hardware components may belong to separate physical systems. For example, processor 420 may include a first processor in a first server and a second processor in a second server. Many other variations and configurations are possible.

According to an embodiment, storage 460 of system 400 may store one or more algorithms, modules, and/or instructions to carry out one or more functions or steps of the methods described or otherwise envisioned herein. Storage 460 may also comprise information or data that can be transferred or otherwise communicated between one or more components of system 400. For example, system 400 may comprise, among other information, first information 462 and/or monitoring information 463. System 400 may also comprise, among other instructions, communication instructions 464 and/or indication or reporting instructions 465.

According to an embodiment, first information 462 may comprise information about one or more healthcare assets associated with the patient in the first environment and/or second environment. For example, the first information may comprise information about one or more healthcare assets associated with or required for the patient. As another example, the first information may comprise information about one or more configurations, settings, or parameters for one or more of the healthcare assets associated with or required for the patient. Other possibilities and embodiments of the first information are described or otherwise envisioned herein.

According to an embodiment, monitoring information 463 may comprise information about the patient, such as health monitoring information, demographic information, medical record information, or any other information. According to an embodiment, the patient is being monitored by one or more healthcare assets configured to obtain information about the patient, such as vital sign monitoring or other information. The information generated by the healthcare assets about the patient may comprise, for example, monitoring information.

According to an embodiment, communication instructions 464 direct the system to communicate information via the composite network between at least a first healthcare environment 470 and a second healthcare environment 480. For example, the communication instructions can direct the system to communicate, via the composite network, a request from the second healthcare environment for the first information and the monitoring information. The communication instructions can also direct the system to communicate, via the composite network, the requested first information and monitoring information in response to the request. The communication instructions can direct the system to communicate, via the composite network, a signal from the second healthcare environment to the first healthcare environment indicating a result of the determining step, such as a determination that the second environment does or does not comprise (i) all necessary healthcare assets for the patient; and/or (ii) all necessary healthcare asset configurations for the patient. Many other communications via the composite network, as directed by the communication instructions 464, are possible.

As an alternative to a composite network, the system may comprise a series of networks, one with monitoring information such as HIPPA data and a second with healthcare asset information. According to an embodiment, these networks may converge or come together either in storage or a processing device which can handle multiple different networks. The system can then process the data to provide a decision as to whether the patient transfer can commence, as describe or otherwise envisioned herein.

According to an embodiment, indication or reporting instructions 465 direct the system to provide an indication, signal, report, or other notification to a healthcare provider in the first environment either: (i) that the second healthcare environment is ready for a patient transfer if the second healthcare environment is determined to be ready for the transfer; or (ii) that the second healthcare environment is not ready for a patient transfer if the second healthcare environment is determined to not be ready for the transfer. Many other indications or reports, as directed by the indication or reporting instructions 465, are possible.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for transferring a patient from a first healthcare environment to a second healthcare environment, comprising:
providing a composite network configured to obtain and communicate: (i) first information about one or more healthcare assets associated with the patient in the first environment; and (ii) monitoring information about the patient;
receiving, at the first environment via the composite network, a request from the second healthcare environment for the first information and the monitoring information, while the patient is in the first healthcare environment;
communicating, via the composite network, the first information and the monitoring information from the first healthcare environment to the second healthcare environment;
configuring, based on at least the received first information, one or more healthcare assets in the second healthcare environment in preparation for a transfer of the patient;
determining whether the second healthcare environment is ready for the transfer of the patient from the first healthcare environment, wherein determining comprises a determination that the second healthcare environment comprises at least: (i) all necessary healthcare assets for the patient; and (ii) all necessary healthcare asset configurations for the patient;
communicating, via the composite network, a signal from the second healthcare environment to the first healthcare environment indicating a result of the determining step; and
receiving, when the second healthcare environment is determined to be ready for the transfer, the patient at the second healthcare environment, or delaying, when the second healthcare environment is determined not to be ready for the transfer, the patient from being transferred to the second healthcare environment.

2. The method of claim 1, wherein the composite network is a single healthcare network configured to communicate the first information and the monitoring information from the first healthcare environment to the second healthcare environment.

3. The method of claim 1, wherein the one or more healthcare assets associated with the patient in the first environment comprise medical equipment.

4. The method of claim 3, wherein the step of configuring one or more healthcare assets in the second healthcare environment in preparation for a transfer of the patient comprises adjusting a setting of one or more pieces of medical equipment in the second healthcare environment.

5. The method of claim 1, wherein the monitoring information about the patient comprises one or more vital signs and/or one or more healthcare treatments for the patient.

6. The method of claim 1, wherein the step of receiving a request from the second healthcare environment for the first information and the monitoring information is in response to a predetermined schedule for the second environment.

7. The method of claim 1, wherein the steps of receiving a request from the second healthcare environment for the first information and the monitoring information through the step of communicating a signal from the second healthcare environment to the first healthcare environment indicating a result of the determining step are automated without human intervention.

8. The method of claim 1, further comprising the step of indicating to a healthcare provider in the first environment either: (i) that the second healthcare environment is ready for a patient transfer if the second healthcare environment is determined to be ready for the transfer; or (ii) that the second healthcare environment is not ready for a patient transfer if the second healthcare environment is determined to not be ready for the transfer.

9. A system configured to enable transfer of a patient from a first healthcare environment to a second healthcare environment, comprising:
a composite network configured to communicate information between a first healthcare environment comprising: (i) first information comprising information about one or more healthcare assets associated with the patient in the first environment; and (ii) monitoring information comprising health information about the patient and the second healthcare environment; and
a processor; configured to: (i) direct communication, via the composite network, of the first information and the monitoring information from the first healthcare environment to the second healthcare environment; (ii) direct configuration, based on the communicated first information, of one or more healthcare assets in the second healthcare environment in preparation for a transfer of the patient; (iii) determine whether the second healthcare environment is ready for the transfer of the patient from the first healthcare environment, wherein determining comprises a determination that the second healthcare environment comprises at least: all necessary healthcare assets for the patient; and all necessary healthcare asset configurations for the patient; and (iv) direct communication, via the composite network, of a signal from the second healthcare environment to the first healthcare environment indicating a result of the determination of readiness or non-readiness.

10. The system of claim 9, further comprising a user interface configured to display, to a healthcare provider in the first environment either: (i) that the second healthcare environment is ready for a patient transfer if the second healthcare environment is determined to be ready for the transfer; or (ii) that the second healthcare environment is not ready for a patient transfer if the second healthcare environment is determined to not be ready for the transfer.

11. The system of claim 9, wherein the composite network is a single healthcare network configured to communicate the first information and the monitoring information from the first healthcare environment to the second healthcare environment.

12. The system of claim 9, wherein the one or more healthcare assets associated with the patient in the first environment comprise medical equipment.

13. The system of claim 12, wherein the step of configuring one or more healthcare assets in the second healthcare environment in preparation for a transfer of the patient comprises adjusting a setting of one or more pieces of medical equipment in the second healthcare environment.

14. The system of claim 9, wherein the monitoring information about the patient comprises one or more vital signs and/or one or more healthcare treatments for the patient.

15. The system of claim 9, wherein the processor is configured to direct communication, via the composite network, of the first information and the monitoring information in response to a predetermined schedule for the first environment and/or the second environment.
